(19) United States
(12) Patent Application Publication
(10) Pub. No.: US 2026/0263426 A1
(43) Pub. Date: Sep. 10, 2026

(54) DIAMIDINE ANALOGS FOR ANTI-INFLAMMATORY USE

(71) Applicant: MONTDOREX INC., Anjou (CA)

(72) Inventor: Terry CHOW, Anjou (CA)

(21) Appl. No.: 19/166,527

(22) PCT Filed: Mar. 28, 2024
(86) PCT No.: PCT/CA2024/050393

§ 371 Date: Sep. 18, 2025

Related U.S. Application Data
(60) Provisional application No. 63/492,661, filed on Mar. 28, 2023.

(51) Int. Cl. A61K 31/4184; A61K 31/4178; A61P 29/00; C07D 233/88; C07D 235/30

(52) U.S. Cl. CPC A61K 31/4184; A61K 31/4178; A61P 29/00; C07D 233/88; C07D 235/30

(57) ABSTRACT

A pharmaceutical composition for use in preventing or treating inflammation in a subject; it has a compound of formula (1): wherein R1 includes one or more cycloalkyl; and R2 includes one or more cycloalkyl, or a pharmaceutically acceptable salt thereof; a method of treating or preventing inflammation in a subject by administrating a therapeutically effective amount thereof to a subject in need thereof.

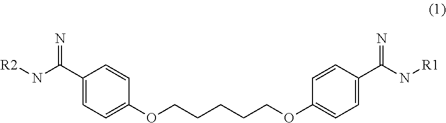

The present application claims priority from U.S. provisional patent application No. 63/492,661 filed on Mar. 28, 2023, incorporated herein by reference.

### TECHNICAL FIELD

The present disclosure relates to treatment and prevention of inflammatory diseases or disorders, and more particularly to the treatment and prevention of inflammatory diseases or disorders through use of a class of compounds, pharmaceutical compositions, that are diamidine analogs.

### BACKGROUND

Pentamidine has been recently studied to bind to lipopolysaccharides (LPS), as described in Wu et al. (2022) Frontiers in Pharmacology, doi: 10.3389/fphar.2022.835081. Lipopolysaccharides are large molecules that include a lipid and a polysaccharide. They are bacterial toxins, found in the outer membrane of gram-negative bacteria. As a result, they cause an inflammatory response in a host. Prolonged exposure to LPS can lead to inflammatory diseases or disorders.

For instance, when LPS is present in the intestines (due to a significant presence of gram negative bacteria), they can lead to inflammatory bowel disease, such as ulcerative colitis or Crohn's Disease. In some instances, LPS may also be present in the liver, the resulting inflammation causing fatty liver disease.

In Wu et al. (2022) Frontiers in Pharmacology, doi: 10.3389/fphar. 2022.835081, it was determined that the ether backbone of pentamidine was involved in binding to LPS, and therefore preventing inflammation resulting from LPS in the subject. When the ether backbone was modified through the addition of an additional oxygen, the anti-inflammatory properties of pentamidine diminished. However, despite its anti-inflammatory properties, pentamidine still demonstrates a significant level of toxicity in a subject, and as a result, due to its toxicity profile, would be inappropriate for the purpose of treating inflammatory diseases or disorders, as the harm would outweigh the benefit, more particularly where pentamidine would have to be taken for prolonged periods in order to treat or prevent inflammation resulting from or relate to the inflammatory disease or disorder.

### SUMMARY

The present disclosure relates to a class of diamidine analogs. At least some of compounds of this class may be suitable for treating LPS-mediated anti-inflammatory disorders in a subject.

Moreover, it has been determined that the toxicity of pentamidine in a subject results from pentamidine's affinity to endo-exonuclease. Endo-exonucleases may function in DNA repair and recombination. For instance, in U.S. Pat. No. 5,324,830 to Resnick et. al. describes the isolation of a DNA segment that codes for an endo-exonuclease, RhoNuc from S. cerevisiae. As a result, inhibiting the endo-exonuclease function in healthy cells using pentamidine could result in toxicity to the subject.

The present disclosure relates to a class of diamidine analog compounds that have been modified to have a reduced binding to endo-exonuclease or preventing binding of the diamidine analog to endo-exonuclease, thereby reducing toxicity to a subject resulting from ingestion of the diamidine analog when compared to pentamidine.

The diamidine analog compounds of the present disclosure can have reduced binding activity to endo-exonuclease activity when compared to pentamidine, thereby reducing their toxicity when administrated to a subject, but still maintain their binding affinity for LPS. As a result, due to their reduced toxicity, these diamidine analog compounds may be used to treat inflammatory diseases or disorders, their toxicity significantly diminished when compared to pentamidine.

In a broad aspect of the present disclosure, the class of diamidine analog compounds has the following structure 1:

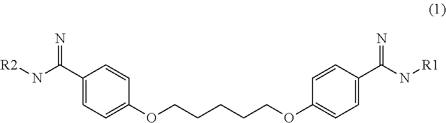

R1 and R2 have been substituted in order to reduce or prevent binding of the diamidine analog with endo-exonuclease. Selection of an appropriate substitute group is required as certain substitute groups at the R1 and R2 positions may instead increase binding affinity with endo-exonuclease, as shown in U.S. Pat. No. 11,103,468 to Chow. Moreover, the size and/or properties of R1 and R2 may not be such that these substitute groups impede with the binding of the compound to LPS through the backbone of the compound.

As a result, R1 and R2 including at least a five-membered cycloalkyl group appears to create sufficient change in the structure of pentamidine (perhaps due at least in part to the bulkiness of the substitute groups including a larger ring structure) to interfere with the binding of the diamidine analog with endo-exonuclease, thereby reducing toxicity to the subject, while maintaining its interactivity with LPS to eliminate LPS, and as a result reduce inflammation.

The diamidine analogs may be ingested, and therefore may be administered orally. By drawing comparisons to known properties and mechanisms of actions of pentamidine, the structure of the diamidine analog compounds is significantly structurally preserved as they pass through the subject's digestive system, and are at least in part absorbed by the liver of the subject. As a result, the diamidine analogs may react with LPS present in the digestive system and/or the liver, thereby eliminating the toxin in the digestive system and/or the liver, reducing inflammation and treating or preventing the disease or disorder related thereto.

A first broad aspect is a compound of formula 1:

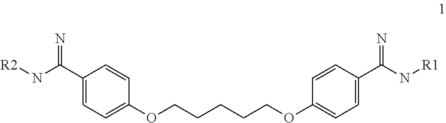

wherein:
   R1 includes one or more cycloalkyl; and
   R2 includes one or more cycloalkyl.

In some embodiments, R1 and R2 may be the same.

In some embodiments, the one or more cycloalkyl of R1 may consist of one cycloalkyl and the one or more cycloalkyl of R2 may consist of one cycloalkyl.

In some embodiments, a ring of the one or more cycloalkyl of R1 may include ring atoms that are selected from C and N, and a ring of the one or more cycloalkyl of R2 includes ring atoms may be selected from C and N.

In some embodiments, the ring of the one or more cycloalkyl of R1 may be partially substituted, and the ring of the one or more cycloalkyl of R2 may be partially substituted.

In some embodiments, the ring of the one or more cycloalkyl of R1 may be aromatic, and the ring of the one or more cycloalkyl of R2 may be aromatic.

In some embodiments, the one or more cycloalkyl of R1 may include imidazole, and wherein the one or more cycloalkyl of R2 may include imidazole.

In some embodiments, the compound may be:

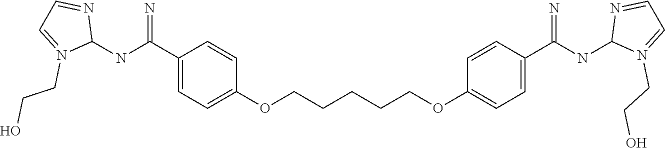

In some embodiments, the compound may be:

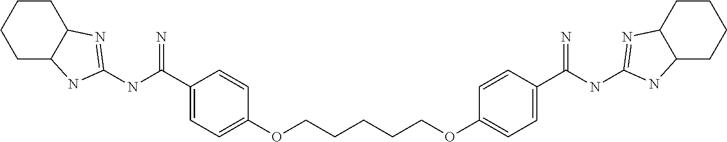 [SMILES: N=C(NC1=NC2CCCCC2N1)c1ccc(OCCCCCOc2ccc(C(=N)NC3=NC4CCCCC4N3)cc2)cc1]

In some embodiments, the compound may be:

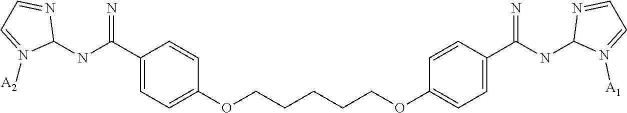

wherein A₁ may be a C₁₋₇ alkyl or a OH—C₁₋₇ alkyl; and A₂ may be a C₁₋₇ alkyl or a OH—C₁₋₇ alkyl.

In some embodiments, A₁ and A₂ may be the same.

In some embodiments, A₁ is a OH—C₁₋₇ alkyl and A₂ may be a OH—C₁₋₇ alkyl.

In some embodiments, the compound may be:

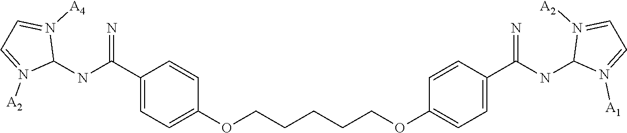

wherein A₁ may be a C₁₋₇ alkyl or a OH—C₁₋₇ alkyl;
A₂ may be a C₁₋₇ alkyl or a OH—C₁₋₇ alkyl;
A₃ may be a C₁₋₇ alkyl, a OH—C₁₋₇ alkyl or a hydrogen; or
A₄ may be a C₁₋₇ alkyl, a OH—C₁₋₇ alkyl or a hydrogen.

In some embodiments, A₁ and A₂ may be the same.

In some embodiments, A is a OH—C₁₋₇ alkyl and A₂ may be a OH—C₁₋₇ alkyl.

In some embodiments, each of A₁ and A₂ may be selected from the group consisting of ethanol, propanol, methanol and isopropanol.

In some embodiments, each of A₃ and A₄ may be selected from the group consisting of ethanol, propanol, methanol and isopropanol.

In some embodiments, A₃ and A₄ may be the same.

In some embodiments, each of A₃ and A₄ may be hydrogen.

In some embodiments, R1 may include at least two N in a monocyclic or polycyclic ring of at least one cycloalkyl of the one or more cycloalkyl, and R2 may include at least two N in a monocyclic or polycyclic ring of at least one cycloalkyl of the one or more cycloalkyl.

Another broad aspect is a pharmaceutical composition for use in preventing or treating inflammation in a subject, comprising a compound of formula 1:

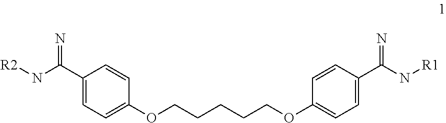

wherein:
   R1 includes one or more cycloalkyl; and
   R2 includes one or more cycloalkyl,
or a pharmaceutically acceptable salt thereof.

In some embodiments, R1 and R2 may be the same.

In some embodiments, the one or more cycloalkyl of R1 may consist of one cycloalkyl and the one or more cycloalkyl of R2 consists of one cycloalkyl.

In some embodiments, a ring of the one or more cycloalkyl of R1 may include ring atoms that are selected from C and N, and a ring of the one or more cycloalkyl of R2 may include ring atoms that are selected from C and N.

In some embodiments, the ring of the one or more cycloalkyl of R1 may be partially substituted, and the ring of the one or more cycloalkyl of R2 may be partially substituted.

In some embodiments, the ring of the one or more cycloalkyl of R1 may be aromatic, and the ring of the one or more cycloalkyl of R2 may be aromatic.

In some embodiments, the one or more cycloalkyl of R1 may include imidazole, and wherein the one or more cycloalkyl of R2 may include imidazole.

In some embodiments, the compound may be:

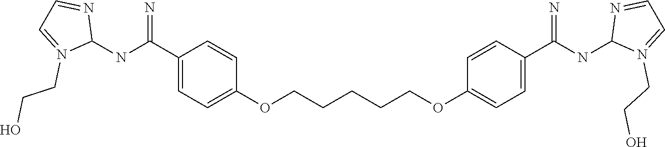

In some embodiments, the compound may be:

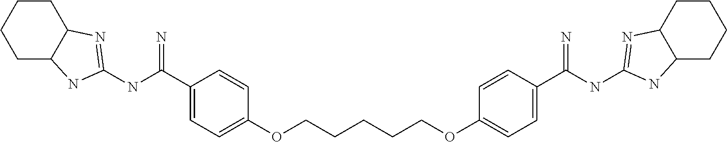 [SMILES: N=C(NC1=NC2CCCCC2N1)c1ccc(OCCCCCOc2ccc(C(=N)NC3=NC4CCCCC4N3)cc2)cc1]

In some embodiments, the inflammation may occur in the intestines, and the pharmaceutical composition may be adapted for oral administration.

In some embodiments, the inflammation may be caused by inflammatory bowel disease.

In some embodiments, the inflammation may occur in the liver, and the pharmaceutical composition may be adapted for oral administration.

In some embodiments, the subject may have fatty liver disease that is related to the inflammation in the liver.

In some embodiments, the compound may be:

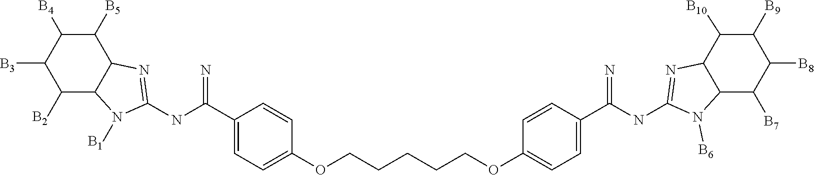

wherein each of B₁, B₂, B₃, B₄, B₅, B₆, B₇, B₃, B₉, B₁₀ may be selected from the group consisting of C₁₋₇ alkyl, OH—C₁₋₇ alkyl and hydrogen.

In some embodiments, one of the groups B₁-B₅ may be C₁₋₇ alkyl or OH—C₁₋₇ alkyl, and the other groups of the groups B₁-B₅ may be H, and one of the groups B₆-B₁₀ may be C₁₋₇ alkyl or OH—C₁₋₇ alkyl, and the other groups of the groups B₆-B₁₀ may be H.

Another broad aspect is a method of treating or preventing inflammation in the at least one of intestines and liver of a subject, comprising administering to a subject in need thereof a therapeutically effective amount of the compound in accordance with the present disclosure, or a pharmaceutically acceptable salt thereof.

In some embodiments, the administration may be performed orally.

In some embodiments, the compound may be found in a slow-dissolving capsule in order to delay release of the compound following oral administration.

In some embodiments, the administration may be performed parenterally.

In some embodiments, the inflammation that is treated or prevented may be in the intestines.

In some embodiments, the inflammation may be caused by or related to inflammatory bowel disease.

In some embodiments, the inflammatory bowel disease may be ulcerative colitis.

In some embodiments, the inflammatory bowel disease may be Crohn's Disease.

In some embodiments, the inflammation that is treated or prevented may be in the liver.

In some embodiments, the inflammation may be caused by or related to fatty liver disease.

Another broad aspect is use of a compound in accordance with the present disclosure for treating or preventing inflammation in a subject.

In some embodiments, the inflammation that is treated or prevented may be in the intestines.

In some embodiments, the inflammation may be caused by or related to inflammatory bowel disease.

In some embodiments, the inflammatory bowel disease may be ulcerative colitis.

In some embodiments, the inflammatory bowel disease may be Crohn's Disease.

In some embodiments, the inflammation that is treated or prevented may be in the liver.

In some embodiments, the inflammation may be caused by or related to fatty liver disease.

Another broad aspect is a method of treating or preventing inflammatory bowel disease in a subject. It includes, upon detecting a presence of inflammatory bowel disease in the subject, administering to the subject in need thereof a therapeutically effective amount of the compound in accordance with the present disclosure, or a pharmaceutically acceptable salt thereof.

In some embodiments, the detecting may be performed by measuring LPS from a gut sample received from the subject.

In some embodiments, the method may include determining an efficacy of treatment by obtaining an indication of inflammation in a bowel of the subject through receiving a subsequent gut sample taken after the administration.

In some embodiments, the indication of inflammation may be determined by measuring a level of LPS in the gut sample.

Another broad aspect is a method of treating or preventing fatty liver disease in a subject. It includes, upon detecting a presence of inflammatory bowel disease in the subject, administering to the subject in need thereof a therapeutically effective amount of the compound in accordance with the present disclosure, or a pharmaceutically acceptable salt thereof.

In some embodiments, the detecting may be performed by measuring LPS from a gut sample received from the subject.

In some embodiments, the method may include determining an efficacy of treatment by obtaining an indication of inflammation in the liver of the subject.

In some embodiments, the indication of inflammation may be determined by measuring a level of LPS in the liver.

### DETAILED DESCRIPTION

The present disclosure relates to a class of diamidine analog compounds that are suitable for treating or preventing an inflammatory disease or disorder in a subject by binding to LPS (an inflammatory agent), while having reduced toxicity to the subject when compared to pentamidine. As a result, due to the reduced toxicity of the class of diamidine analog compounds, the class of diamidine analog compounds is suitable for treating or preventing the inflammatory diseases or disorders.

The diamidine analog compounds maintain the ether backbone of pentamidine that has been demonstrated to confer to pentamidine its ability to target and bind to LPS, thereby deactivating or eliminating LPS, and reducing the inflammation in the subject resulting therefrom. However, the diamidine analog compounds include cycloalkyl as end substitutions, the cycloalkyl reducing or preventing binding of the diamidine analog to endo-exonuclease, and thereby reducing the toxicity relating thereto.

The diamidine analog compounds in accordance with the present disclosure have the following structure (1):

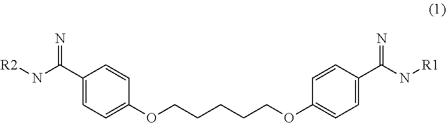

R1 and R2 are each one or more cycloalkyl for reducing the binding of the compound with endo-exonuclease when compared to pentamidine.

Due to pentamidine's, and therefore also the diamidine analog compounds of the present disclosure's, capacity for oral administration and significant preservation of integrity throughout the digestive system of a subject, the diamidine analog compounds of the present disclosure are appropriate for treating or preventing inflammatory diseases and/or disorders located in the intestines (such as inflammatory bowel disease—e.g. Crohn's disease; ulcerative colitis). Moreover, as the diamidine analog compounds, drawing a comparison to pentamidine, equally travel to the liver, the diamidine analog compounds may also treat and/or prevent inflammatory diseases and/or disorders that are located in the liver (such as fatty liver disease).

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

### Definitions

The term “C₁₋₇ alkyl”, as used herein, refers to a straight or branched hydrocarbon chain radical consisting of carbon and hydrogen atoms, containing no unsaturation, having one to seven carbons, and which is attached to the rest of the molecule by a single bond. Examples of C₁₋₇ alkyls include, but are not limited to: methyl, ethyl, n-propyl, iso propyl, butyl, isobutyl, etc.

The term “C₂₋₇ alkene”, as used herein, refers to a straight or branched hydrocarbon chain radical consisting of carbon and hydrogen atoms, containing one or more double bonds, having two to seven carbons, and which is attached to the rest of the molecule by a single bond. Examples of C₁₋₇ alkenes include, but are not limited to: ethylene, propylene, butylene, etc.

The term “C₂₋₇ alkyne”, as used herein, refers to a straight or branched hydrocarbon chain radical consisting of carbon and hydrogen atoms, containing one or more triple bonds, having two to seven carbons, and which is attached to the rest of the molecule by a single bond. Examples of C₁₋₇ alkyne include, but are not limited to: acetylene, propyne, etc.

The term “cycloalkyl” as used herein, refers to a five to ten membered monocyclic or polycyclic ring, saturated, partially saturated or unsaturated, where the ring atoms are selected from N, O, S and C. Examples of cycloalkyl include, but are not limited to, azetidinyl, tetrahydrofuran, dihydrofuran, dioxane, morpholine, etc. A cycloalkyl may be optionally substituted by one to five substituents independently selected from, for instance, the group consisting of hydroxy, thiol, cyano, nitro, a OH—C₁₋₇ alkyl, linear or branched, sulfonyl, halogen or amino.

The term “gut sample”, as used herein, refers to a sample that has originated from a subject's gastrointestinal track. In some examples, the gut sample may be specific to the colon. In other examples, the gut sample may be specific to the ascending colon or the descending colon. A gut sample may be a stool sample or any other non-invasive sample that has originated from the subject's gut. In some examples, the gut sample may involve obtaining a mucosal luminal interface (MLI) sample from the subject. In some examples, the gut sample may be a biopsy sample taken from a subject, obtained during, for instance, a colonoscopy.

The term “C₁₋₇ alkoxy”, as used herein, refers to a radical of formula —ORₐ where Rₐ is a C₁₋₇ alkyl radical as generally defined above. Examples of C₁₋₇ alkoxy include, but are not limited to: methoxy, propoxy, butoxy, pentoxy, etc.

The term “OH—C₁₋₇ alkyl”, as used herein, refers to a C₁₋₇ alkyl radical, where one or one of the hydrogen atoms of the C₁₋₇ alkyl is replaced by “OH”. Exemplary OH—C₁₋₇ alkyl include, but are not limited to, hydroxy-methyl, hydroxy-ethyl, 2-hydroxy propyl, 2-hydroxy-butyl, etc.

The term “amino-C₁₋₇ alkyl”, as used herein, refers to a C₁₋₇ alkyl radical, where one of the hydrogen atoms of the C₁₋₇ alkyl is replaced by an amino group. Exemplary amino-C₁₋₇ alkyls include, but are not limited to, amino-methyl, amino-ethyl, amino-propyl, 2-amino-butyl, etc.

The term “halogen”, as used herein, refers to bromo, chloro, fluoro or iodo.

The term “halogen-C₁₋₇ alkyl”, as used herein, refers to a C₁₋₇ alkyl, where one or more of the hydrogens are replaced by a halogen.

The term “inflammatory bowel disease”, as used herein, refers to ulcerative colitis (UC), subjects with Crohn's disease (CD) and/or subjects with IBD-unclassified (IBD-U).

The term “inhibit”, as used herein, refers to the reduction or suppression of a given condition, symptom, disease or disorder, or a reduction in the baseline activity of a biological activity or process.

The term “measuring”, as used herein, a protein sample as used herein, refers to conducting an analysis of a sample to determine a protein expression level (or relative protein expression level) using such techniques as an immunoassay (e.g. ELISA), semi-quantitative immunoblotting, mass spectrometry, and other techniques that are known in the art to quantitatively and/or qualitatively analyze the contents of a sample obtained from a subject.

The term “pharmaceutical composition”, as used herein, refers to a compound in accordance with the present disclosure, or a pharmaceutically acceptable salt thereof, in a form suitable for oral administration or parenteral administration.

The term “pharmaceutically acceptable carrier”, as used herein, refers to a substance useful in the preparation of a pharmaceutical composition and includes, for instance, one or more of diluents, surfactants, preservatives, buffering agents, isotonic agents, salts, excipients, lubricants, wetting agents, flavoring, etc.

The term “prevent”, “preventing” or “prevention”, as used herein, refers to the prophylactic treatment of a disease or disorder, or delaying the onset or progression of a disease or disorder.

In the present disclosure, by “subject”, it is meant a mammal, such as a human. The term “subject” should not bring on any limitations as to the sex or age.

The term “treat”, “treating” or “treatment”, as used herein, refers to alleviating or improving the outcome of the subject with regard to a given disease or disorder, which may be quantifiable by improving at least one physical parameter, ailment or biomarker of the subject with the disease or disorder.

### Exemplary Synthesis—Reaction Scheme 1

The diamidine analog compounds of the present disclosure may be produced through organic synthesis methods as are known in the art, with reference to the following reaction schemes and examples.

Starting materials, building blocks, reagents, acids, bases, dehydrating agents, solvents, and so forth, that are used to synthesize the present compounds may either be commercially available or produced using known techniques of organic synthesis.

As used herein, the term “salt” or “salt” refers to an acid salt or base salt of a compound of the present disclosure. The term “pharmaceutically acceptable salts” refers to salts that retain the biological function and properties of the compounds of the present disclosure. For instance, the compounds of the present disclosure are capable of forming acid and/or base salts as a result of amino and/or carboxyl groups (or similar groups).

A pharmaceutically acceptable acid salt can be formed using an inorganic acid (e.g. hydrochloric acid, sulfuric acid, nitric acid, etc.) and/or an organic acid (e.g. acetic acid; oxalic acid; succinic acid; etc.).

A pharmaceutically acceptable base salt can be formed using an inorganic base (e.g. sodium hydroxide, potassium hydroxide, magnesium hydroxide, etc.) and/or an organic base (e.g. isopropylamine, cholinate, lysine, etc.)

Any formula provided herein is intended to also represent unlabeled forms or isotopically labelled forms.

Intermediates and final produces can be purified using known methods (e.g. such as chromatographic methods; distribution methods; re-crystallization, etc.)

A method for synthesis of compounds of Formula (1) are provided in Scheme 1 below:

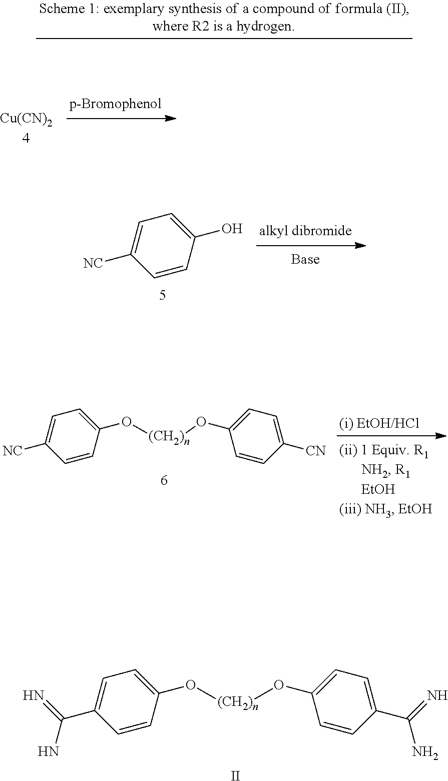

The synthetic strategy for the preparation of a compound of formula (1) consists of using p-bromophenol, and a nitrile moiety at the para position can be substituted to yield compound 5. The dinitrile 6 can be obtained by treatment of at least two equivalents of compound 5 with an appropriate alkyl dibromide or an alkyl dihalide or an alkyl reagent with two leaving groups, in the presence of a base to yield compound 6. Further reaction of 6 to convert the dinitrile into Species 1 can be accomplished by treatment with ethanol in the presence of HCl, followed by treatment with one equivalent of an appropriate amine (R2NH2), and then with ammonia in ethanol.

### Exemplary Synthesis—Reaction Scheme 2

The synthetic strategy for the preparation of a compound of formula (1), consists of using compound 6 to convert the dinitrile into Species 1 can be accomplished by treatment with ethanol in the presence of HCl, followed by treatment with one equivalent of an appropriate amine (R₁NH₂), and one equivalent of an appropriate amine (R₂NH₂).

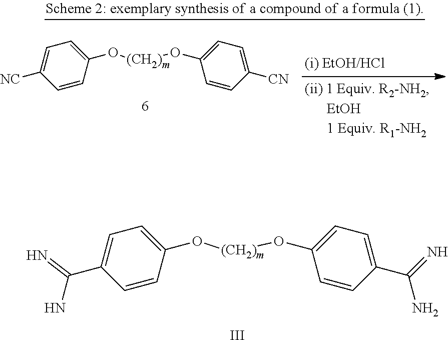

### Exemplary Preparation of Pharmaceutical Compositions:

Pharmaceutical compositions of the present disclosure may be prepared as pharmaceutically acceptable salts by reacting a base form of the compound with a pharmaceutically acceptable acid. In some examples, instead, a free acid form of the compound may be reacted with a base.

The base form of the compound or the acid form of the compound can be prepared from the corresponding base salt form or acid salt form.

Any asymmetric atom (e.g. carbon) of the compound of the present disclosure may be prepared in a racemic or enantiomerically enriched form, for example (R) or(S), or (RS) configuration. In some embodiments, each enantiomeric atom has at least 50% enantiomeric excess. In some embodiments, each enantiomeric atom has at least 60% enantiomeric excess. In some embodiments, each enantiomeric atom has at least 70% enantiomeric excess. In some embodiments, each enantiomeric atom has at least 80% enantiomeric excess. In some embodiments, each enantiomeric atom has at least 90% enantiomeric excess. Substituents of unsaturated bonds may be present in -cis or -trans configuration.

Any resulting mixtures of stereoisomers can be separated based on physiochemical differences between its constituents, e.g., using chromatography and/or fractional crystallization.

The present disclosure further includes any variant of the present processes, where an intermediate that is obtainable at any stage of the process may be used as a starting material. It will be understood that compounds and intermediates disclosed herein can be converted between one another used techniques that are known in the art. When the production of certain materials in not particularly described herein, it will be understood that their synthesis may be performed in accordance with teachings as are known in the art.

It will be understood that the above schemes are only representative of methods of preparation of the present compounds, and that other well methods may be performed instead to synthesizes the compounds of the present disclosure.

The following exemplary study is provided to enable the skilled person to better understand the present disclosure. As it is but illustrative and representative examples, it should not limit the scope of the present disclosure, only added for illustrative and representative purposes. It will be understood that other exemplary studies may be used to further illustrate and represent the present disclosure without departing from the present teachings.

### Exemplary Study 1:

The inhibition of endo-exonuclease has been measured using pentamidine and a pentamidine analog where the center site of pentamidine (the carbon backbone) has been altered. Endo-exonuclease inhibition has been tested in-vitro. The results are presented below, in Table 1:

TABLE 1
inhibition of endo-exonuclease activity following the modification of the center carbon backbone.

| | | | HCT- | |
|---|---|---|---|---|
| | | | 116 | EE |
| | Molecular | | IC50 | IC50 |
| Name | Weight | Structure | (μM) | (μM) |
| Pentamidine HCl | 340.42 g/mol Salt: 413.34 g/mol | 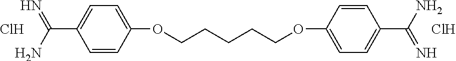 [SMILES: Cl ; N=C(N)c1ccc(OCCCCCOc2ccc(C(=N)N)cc2)cc1 ; Cl] | 107 | 53.1 |
| Decamidine- isethionate | 410.55 g/mol Salt: 662.82 g/mol | 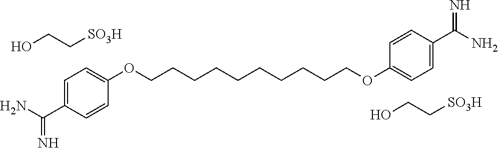 | 6.2 | 23.2 |

As such, it appears from the results presented in Table 1 that the modification of the center carbon backbone of pentamidine does not eliminate endo-exonuclease activity. As endo-exonuclease inhibition is related to cytotoxicity of the compound to the patient, modification of this center backbone of pentamidine would not at least significantly reduce cytotoxicity when administering this compound to a subject for purposes of treating an inflammatory disorder. Instead, endo-exonuclease inhibition by pentamidine is associated with the structure of the ends of pentamidine (by making a substitution on either end of the pentamidine molecule at the nitrogen joined by a single bond to the neighbouring carbon atom—where the hydrogen bonded to the nitrogen is substituted for another group). A further determination has been made that a substitution at these R1 and R2 positions of pentamidine with a cycloalkyl, preferably with one or more nitrogen atoms, leads to a reduction of inhibition of endo-exonuclease activity by the pentamidine analog, when compared to pentamidine, while preserving the compounds binding affinity to LPS. For instance, it has been determined that suitable substitute groups at the R1 and R2 positions may be either 3a,4,5,6,7,7a-hexahydrobenzimidazol or N-hydroxyethylimidazol.

### Discussion

It has been discovered that an addition of a bulky group to the R1 and R2 position of the following diamidine analog reduces or prevents binding of pentamidine to endo-exonuclease:

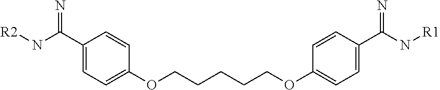

For instance, it has been discovered that having both R₁ and R2 defined as:
3a,4,5,6,7,7a-hexahydrobenzimidazol; or
N-hydroxyethylimidazol,
   can prevent the binding of the diamidine analog to endo-exonuclease, while preserving its binding to LPS. As a result, these diamidine analogs demonstrate significant reduced toxicity as a result of its reduced or prevented binding to endo-exonuclease (where endo-exonuclease activity has been correlated to toxicity in humans), while acting as an anti-inflammatory by binding to LPS.

However, it is postulated that including a bulky group alone in the R1 and R2 position may be insufficient to reduce binding of the diamidine analog to endo-exonuclease, and as a result, reducing toxicity. This has been exemplified in U.S. Pat. No. 11,103,468 to Chow, where having phenyl as R1 and R2 substitutions instead increased binding affinity of a diamidine analog to endo-exonuclease, when compared to pentamidine.

As a result, it is reasoned that including a cycloalkyl in the R1 and R2 positions with one or more nitrogens, or an atom from the same column of the periodic table as nitrogen (such as phosphorus) is what contributes to reducing the binding affinity of the diamidine analog to endo-exonuclease, thereby reducing toxicity following administration of the diamidine analog to a subject.

In Zhao E et al (2017) Hepatology 66:922-935, it has been shown that pentamidine reduces hepatic triglycerides, serum ALT, and body weight and food intake in mice. This study demonstrates that pentamidine has been shown to reduce non-alcoholic fatty liver disease in mice. However, due to the interaction between pentamidine and endo-exonuclease, which could lead to cytotoxicity in the patient when treating fatty liver disease, reduction of the endo-exonuclease inhibition by the administered compound would be advantageous to reduce cytotoxicity. This may be achieved by making substitutions at the R1 and R2 positions of pentamidine, where more particularly these substitutions include one or more ring structures (in some examples, preferably, including at least one nitrogen atom in the ring structure). It has been shown that cycloalkyl substitutions at the R1 and R2 positions of pentamidine lead to a reduction in endo-exonuclease while preserving the binding affinity of the compound to LPS.

Moreover, it has further been discovered that substitutions in the R1 and R2 position do not affect the binding affinity of the diamidine analog to LPS, therefore preserving its anti-inflammatory properties despite the R1 and R2 substitutions. However, maintaining the carbon-ether backbone of the diamidine analog has been proven to be important to preserve the LPS binding of the diamidine analog.

Aromaticity of the rings in R1 and R2 does not appear to be necessary in order to reduce or eliminate binding of the diamidine analog to endo-exonuclease. However, having one or more unsaturated bonds in the rings of R1 and R2 of the diamidine analogs does appear to at least contribute to reducing binding activity of the diamidine analog to endo-exonuclease.

### Exemplary Method of Treating Inflammatory Bowel Disease:

An exemplary use of the diamidine analog compounds of the present disclosure is for the treatment and/or prevention of inflammatory caused by or related to inflammatory bowel disease. Reference is made herein to an exemplary method of treating inflammatory bowel disease using the class of diamidine analog compounds described herein.

A presence of inflammatory bowel disease may be determined for the subject. The presence of inflammatory bowel disease may be detected by measuring one or more biomarkers (e.g. calprotectin; LPS; etc.) from one or more gut samples received from the subject, symptoms experienced by the subject, etc., as is known in the art for the purpose of detecting inflammatory bowel disease in a subject.

Upon detection or determination of presence of inflammatory bowel disease in a subject, administration of a therapeutically-effective amount of one or more diamidine analog compounds in accordance with the present teachings may be performed. In some embodiments, the diamidine analogs may be administrated orally. In some instances, a capsule containing the one or more diamidine analogs may be a slow-release capsule to, e.g., delay release of the one or more diamidine analog compounds until the capsule travels to the colon.

In some instances, the diamidine analog compounds may be administrated parenterally.

Following administration of the therapeutically-effective amount of one or more diamidine analog compounds, gut samples from the subject may be subsequently taken to determine if the inflammatory bowel disease is effectively being treated (where biomarkers may be measured from these gut samples, and compared to the corresponding results derived from the initial gut sample).

### Exemplary Method of Treating Fatty Liver Disease:

An exemplary use of the diamidine analog compounds of the present disclosure is for the treatment and/or prevention of inflammation caused by or related to fatty liver disease. Reference is made herein to an exemplary method of treating fatty liver disease using the class of diamidine analog compounds described herein.

A presence of fatty liver disease may be determined for the subject. The presence of fatty liver disease may be detected by measuring one or more biomarkers (e.g. apolipoprotein A1, apolipoprotein B, leptin, adiponectin, free fatty acids, LPS, ghrelin tumour necrosis factor-alpha, etc.) from one or more samples taken from the subject, determined from symptoms recounted by the subject, etc., as is known in the art for the purpose of detecting fatty liver disease in a subject.

Upon detection or determination of a presence of fatty liver disease in a subject, administration of a therapeutically-effective amount of one or more diamidine analog compounds in accordance with the present teachings may be performed. In some embodiments, the diamidine analogs may be administrated orally. In some instances, a capsule containing the one or more diamidine analogs may be a slow-release capsule to, e.g., delay release of the one or more diamidine analog compounds.

In some instances, the diamidine analog compounds may be administrated parenterally.

Following administration of the therapeutically-effective amount of one or more diamidine analog compounds, subsequent samples from the subject may be taken to determine if the fatty liver disease is effectively being treated (where biomarkers may be measured from these samples, and compared to the results of the initial sample received from the subject).

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings to provide useful amidine and diamidine compounds and methods of treating cancer using the same.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

All features disclosed in the description and/or the claims are intended to be disclosed separately and independently from each other for the purpose of original written disclosure, as well as for the purpose of restricting the claimed subject matter, independent of the compositions of the features in the embodiments and/or the claims. In addition, all value ranges or indications of groups of entities are intended to disclose every possible intermediate value or intermediate entity for the purpose of original written disclosure, as well as for the purpose of restricting the claimed subject matter.

What is claimed is:

1. A compound of formula 1: 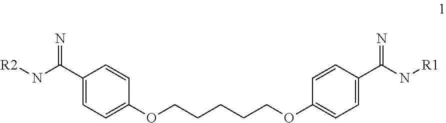
wherein:
R1 includes one or more cycloalkyl; and
R2 includes one or more cycloalkyl.

2. The compound in accordance with claim 1, wherein R1 and R2 are the same.

3. The compound in accordance with claim 1 or claim 2, wherein the one or more cycloalkyl of R1 consists of one cycloalkyl and the one or more cycloalkyl of R2 consists of one cycloalkyl.

4. The compound in accordance with any one of claims 1 to 3, wherein a ring of the one or more cycloalkyl of R1 includes ring atoms that are selected from C and N, and a ring of the one or more cycloalkyl of R2 includes ring atoms that are selected from C and N.

5. The compound in accordance with claim 4, wherein the ring of the one or more cycloalkyl of R1 is partially substituted, and the ring of the one or more cycloalkyl of R2 is partially substituted.

6. The compound in accordance with claim 5, wherein the ring of the one or more cycloalkyl of R1 is aromatic, and the ring of the one or more cycloalkyl of R2 is aromatic.

7. The compound in accordance with claim 6, wherein the one or more cycloalkyl of R1 includes imidazole, and wherein the one or more cycloalkyl of R2 includes imidazole.

8. The compound in accordance with claim 7, wherein the compound is:

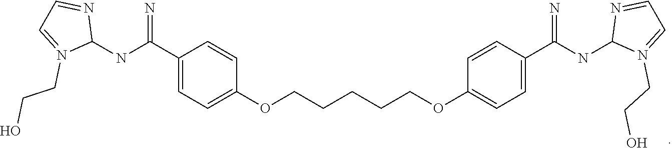

9. The compound in accordance with any one of claims 1 to 5, wherein the compound is:

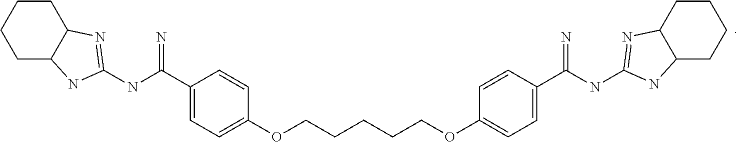 [SMILES: N=C(NC1=NC2CCCCC2N1)c1ccc(OCCCCCOc2ccc(C(=N)NC3=NC4CCCCC4N3)cc2)cc1]

10. A pharmaceutical composition for use in preventing or treating inflammation in a subject, comprising a compound of formula 1: 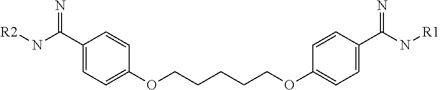
wherein:
R1 includes one or more cycloalkyl; and
R2 includes one or more cycloalkyl,
or a pharmaceutically acceptable salt thereof.

11. The pharmaceutical composition in accordance with claim 10, wherein the one or more cycloalkyl of R1 consists of one cycloalkyl and the one or more cycloalkyl of R2 consists of one cycloalkyl.

12. The pharmaceutical composition in accordance with claim 10 or claim 11, wherein a ring of the one or more cycloalkyl of R1 includes ring atoms that are selected from C and N, and a ring of the one or more cycloalkyl of R2 includes ring atoms that are selected from C and N.

13. The pharmaceutical composition in accordance with claim 12, wherein the ring of the one or more cycloalkyl of R1 is partially substituted, and the ring of the one or more cycloalkyl of R2 is partially substituted.

14. The pharmaceutical composition in accordance with claim 13, wherein the ring of the one or more cycloalkyl of R1 is aromatic, and the ring of the one or more cycloalkyl of R2 is aromatic.

15. The pharmaceutical composition in accordance with any one of claims 10 to 14, wherein the one or more cycloalkyl of R1 includes imidazole, and wherein the one or more cycloalkyl of R2 includes imidazole.

16. The pharmaceutical composition in accordance with claim 15, wherein the compound is:

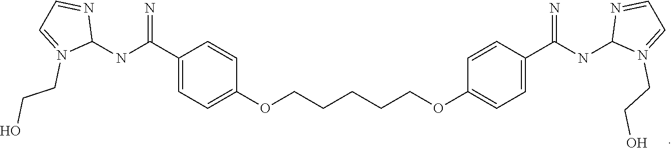

17. The pharmaceutical composition in accordance with any one of claims 10 to 15, wherein the compound is:

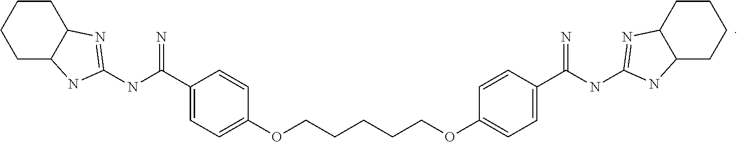 [SMILES: N=C(NC1=NC2CCCCC2N1)c1ccc(OCCCCCOc2ccc(C(=N)NC3=NC4CCCCC4N3)cc2)cc1]

18. The pharmaceutical composition in accordance with any one of claims 10 to 17, wherein the inflammation occurs in the intestines, and the pharmaceutical composition is adapted for oral administration.

19. Use of a compound in accordance with any one of claims 1 to 9 for treating or preventing inflammation in a subject.

20. The method in accordance with claim 19, wherein the inflammation is caused by or related to fatty liver disease.
